(19) United States
(12) Patent Application Publication
(10) Pub. No.: US 2014/0134566 A1
(43) Pub. Date: May 15, 2014

(54) Dental Prosthesis With Reference Points For Imaging When Fabricating A Denture

(71) Applicants: Robert Lemke, San Antonio, TX (US); John Marion Tharpe, JR., Albany, GA (US)

(72) Inventors: Robert Lemke, San Antonio, TX (US); John Marion Tharpe, JR., Albany, GA (US)

(21) Appl. No.: 13/802,200

(22) Filed: Mar. 13, 2013

Related U.S. Application Data
(63) Continuation-in-part of application No. 13/676,887, filed on Nov. 14, 2012.

(51) Int. Cl. A61C 13/00; A61C 9/00; A61B 19/00; A61C 8/00

(52) U.S. Cl. CPC A61C 13/0004; A61C 8/0068; A61C 9/0053; A61B 19/54
USPC 433/29; 433/173

(57) ABSTRACT

A dental prosthesis apparatus with reference points for overlapping of multiple images to form a composite image, in which a pair of implant assemblies each attaches to a clip assembly having first and second opposing rail clips secured together with a fastener while holding a portion of an elongated rail that extends between the implant assemblies, and the rail defines a differentiation characteristic for use as reference points for matching of a plurality of images in overlapping relation for forming a composite image. A method of providing a dental prosthesis with reference points for imaging is disclosed.

### TECHNICAL FIELD

The present invention relates to dental prosthesis and fabrication. More particularly, the present invention relates to dental prosthesis readily fabricated chair side of a patient with reference points for digital imaging.

### BACKGROUND OF THE INVENTION

In recent years, dental implants have become a viable option for both partial and full prosthesis whereby a missing tooth, or multiple missing teeth, are replaced with an artificial fabricated substitute. There are at least several manufacturers providing dental prosthesis devices. The dental prosthesis technology uses implants that seat in bone structure of the jaw, and a variety of secondary connections referred to as abutments attach rigidly to the implants. Bridges attach to the abutments, and restorative teeth attach in spaced-relation to the bridges.

Generally, a dental surgeon places the implants within bone. The implant location is selected to facilitate acceptance of the implant by bone structure while avoiding weak bone sections, nerves, and sinus cavities. The implants may be disposed at oblique angles in order to seat a longer implant while forming strong implant-to-bone connections. The implants integrate with the bone through bone growth, typically over a several month period. The implants become a foundation that supports the bridges and restorations. An abutment is selected from a variety of abutments having differing characteristics (length, angle, cross-sectional width). The abutment attaches rigidly to the implant for alignment of the bridge.

The abutments are installed upon satisfactory seating of the implant within the bone. Because implant positions are limited to locations of good bone, the abutments provide a gross adjustment of misaligned or off-axis direction of placement of the implant. Multiple implants may be connected together, or splinted, for cross-arch stabilization during the integration process, such as attaching a temporary prosthesis prior to full integration.

A temporary prosthesis, or bridge of teeth, is fabricated for use during integration. The temporary prosthesis is typically made of acrylic, for short term use, as such material is subject to fracture. Thus, temporary prosthesis may not allow for full occlusion whereby the patient may chew food properly.

Upon satisfactory healing of the implant/bone root form, the patient is attended by a prosthodontist for fabrication of a permanent prosthesis. The healing process may be several or more months. With implant/bone integration, an impression is formed of the patient's maxillary structure. The impression is used by a laboratory for fabrication of a rigid framework to support the denture or bridge of teeth. Traditional casting may be used to form the denture, or more recently, digital scanning and CAD/CAM technology may be used. This latter technology features milling a titanium block to form a unique framework. This is specialized machining and there are a limited number of experienced product providers. The labor is time consuming and materials expensive. Once the temporary bridge is replaced with the permanent bridge, the patient is provided with an excellent prosthesis.

However, there are drawbacks limiting the desirability of such permanent prosthesis. Should an implant fail, or move, the milled bar must be completely discarded. The fabrication process recommences with the impression/scanning/milling process repeated. The milled parts are not retrievable for re-use. Generally, the process is time consuming. The time required may span from several months to years for the final permanent prosthesis to be installed. Further, the process requires services of multiple professionals working at various locations remote from each other. As an alternative, a technical company has attempted to shorten the time required by housing the three required professional (surgeon, prosthodontist and dental laboratory technician) in a single complex.

Further, there are drawbacks with these systems for fabrication of prosthesis. The support bars need to be horizontal. Due to positioning variances and angular alignment of the implants, each abutment requires separate height alignment. Further, the abutments require different angular alignment in order to orient the support member vertical for engaging the denture. The present systems further do not readily permit fabrication of the front retaining bar that aligns with the parabolic-shaped dental arch. The rigid assembly of the denture is not removable during fabrication. Also, in the event of moving of the implant, the components used in the denture are lost because of the special design configured for the particular position of the implant. Components are not retrievable and reusable in the event of movement of an implant. Also, the implants provide point-to-point support for the prosthesis and are unsatisfactory for including a cantilever extended portion so as to enlarge the occlusal chewing surface.

Traditionally, 3-dimensional models of teeth, bone and soft tissues of the mouth are formed with a mixture of two-part impression materials. The mixture is placed around teeth and their adjacent structures in the mouth. Upon setting, a flexible mold is removed and used for preparing restorations. There are drawbacks to using such flexible impression, including unsatisfactory tolerances. As a consequence, the use of digital imaging for creating restorations is increasing in dental surgeries.

Digital imaging uses technologies based on CMOS and CCD imaging devices. CMOS devices provide re-usable media for imaging in a manner similar to radiography imaging of film negatives. CCD devices image optically using such media as video, laser or optical images. The CCD device communicates with a computer software application executed on a computer. The dental technician moves the CCD imaging device over the lingual, occusal, and labial or buccal surfaces of the teeth. The software application driving the CCD imaging device is configured to make either single images upon actuation by the dental technician or to make a multiple number of images during the scanning process, typically up to 30 images per second (video mode) but systems are available that generate multiple times this number.

The images communicate to the computerized application that receives the images, stores the images for display and analysis, and displays the images. The application includes a compiler that matches a plurality of the images in overlapping relation to form a composite or 3-dimensional composite image of the patient's teeth and gingiva structures. The 3-dimensional data of the composite image may be manipulated in a computer aided design and mapping system for selective display of the image of the teeth and gingiva on a monitor or for use in preparing restorations. The compiler uses distinctive features imaged from the teeth and adjacent soft and hard tissues to match the overlapping images. However, imaging of soft tissue alone (gingiva), such as with multiple missing teeth, is difficult because distinguishing features are typically not present and the images of the gingiva take on a uniform indistinctive appearance.

Accordingly there is a need in the art for providing reference points for matching multiple overlapping images from digital scanning of gingiva structures for restoration surgery. It is to such that the present invention is directed.

### SUMMARY OF THE INVENTION

The present invention meets the need in the art by providing a dental prosthesis apparatus with reference points, comprising at least a pair of implant assemblies, each implant for selective imbedding in a maxillary bone structure and defining a threaded bore and at least a pair of clip assemblies. Each clip assembly comprises a first rail clip; an opposing second rail clip; a fastener for securing the first rail clip and the second rail clip in spaced relation; and a threaded member for securing the clip assembly to the implant assembly by engaging the threaded bore. A rail extends between the pair of clip assemblies and is disposed with a respective portion between the first rail clip and the second rail clip of each of the clip assemblies. The clip assembly, being secured with the threaded member connected to the implant assembly, rigidly holds the rail between the first rail clip and the second rail clip. The rail defines a differentiation characteristic for use as reference points for matching a plurality of images in overlapping relation for a composite image by an image compiler.

In another aspect, the present invention provides a method of positioning reference points in a dental prosthesis apparatus for imaging of a dental patient's mouth and gingiva for restorative surgery, comprising the steps of:

(a) imbedding in a maxillary bone structure at least a pair of implant assemblies, each implant defining a threaded bore;

(b) attaching at least a pair of clip assemblies, each clip assembly attaching to a respective implant assembly, each clip assembly having a first rail clip and an opposing second rail clip;

(c) extending a rail having a differentiation characteristic between the first rail clip and the second rail clip in each clip assembly; and

(d) securing the first rail clip and the second rail clip together in the respective clip assembly to hold the rail rigidly therebetween;

whereby the rail having the differentiation characteristic, defines thereby reference points for matching a plurality of images in overlapping relation for a composite image by an image compiler.

Objects, advantages, and features of the present invention will become readily apparent upon a reading of the detailed description in reference to the drawings and the appended claims.

### BRIEF DESCRIPTION OF THE DRAWINGS

FIG. 1 illustrates a full arch assembly of a dental prosthesis in accordance with the present invention.

FIG. 2 is an exploded view of a prosthesis apparatus used in the dental prosthesis illustrated in FIG. 1.

FIG. 3 is a detailed prospective view of an upper rail clip used in the illustrated embodiment of the dental prosthesis shown in FIG. 1.

FIG. 4 is a detailed respective view of a lower rail clip and spacer column used in the illustrated embodiment of the dental prosthesis shown in FIG. 1.

FIG. 5 is a prospective view of a rail member for being received by the lower rail clip and the upper rail clip shown in FIGS. 3 and 4 for the dental prosthesis shown in FIG. 1.

FIG. 6 is a prospective exploded view of an alternate embodiment of a rail for use in a dental prosthesis in accordance with the present invention.

FIG. 7 illustrates a denture molded of a conventional material with a plurality of teeth for containing the prosthesis apparatus illustrated in FIG. 1.

FIG. 8 illustrates a dental prosthesis with a first embodiment of a differentiation rail having a plurality of indicator members for reference points for digital imaging of a patient's mouth and soft/hard tissue structure for restoration surgery.

FIG. 9 illustrates a second embodiment of a differentiation rail with an irregular differentiated surface for defining reference points for digital imaging of a patient's mouth and gingiva structure for restoration surgery.

FIG. 10 illustrates a third embodiment of a differentiation rail with an irregular texture field for defining reference points for digital imaging of a patient's mouth and soft/hard tissue structure for restoration surgery.

FIG. 11 illustrates a fourth embodiment of a differentiation rail that defines a plurality of shaped openings along the rail for reference points for digital imaging of a patient's mouth and soft/hard tissue structure in restoration surgery.

### DETAILED DESCRIPTION

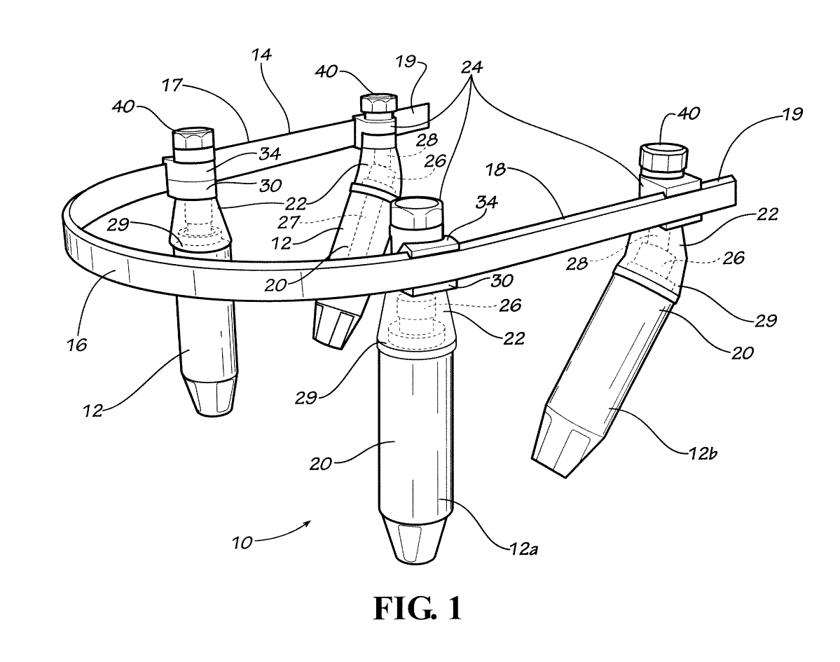

With reference to the drawings, in which like parts have like identifiers, FIG. 1 illustrates a full arch assembly of a dental prosthesis 10 in accordance with the present invention. The prosthesis 10 in the illustrated embodiment includes four implant assemblies 12 and a rail 14 that defines a parabolic-shaped dental arch portion 16 and opposing extending posterior arch portions 17, 18. In the illustrated embodiment, a portion 19 of the rail 14 extends as a cantilever longitudinally posterior of a posterior implant assembly 12b.

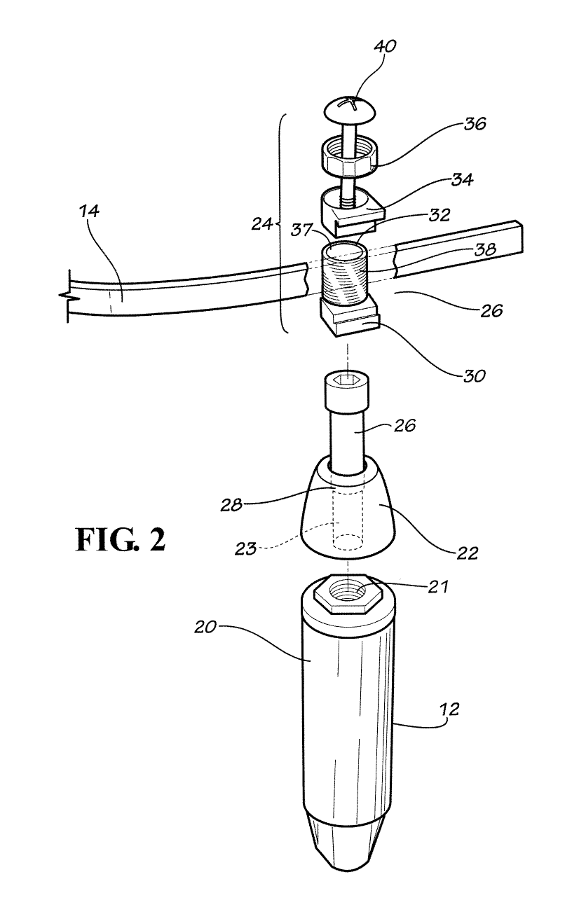

With reference to FIGS. 1 and 2, each implant assembly 12 includes a dental implant 20, an abutment 22, and a clip assembly 24 (shown in detailed exploded view in FIG. 2). The clip assembly 24 secures the rail 14 to the abutment 22 of the implant 20. The implant 20 and the abutment 22 are conventional members provided by dental prosthesis providers. The implant 20 seats in a bone structure of a patient for the dental prosthesis 10. The implant 20 defines an internal threaded bore 21. The abutment 22 defines a first passageway 23. The abutment 22 seats on the upper end of the implant 20. An abutment screw 26 extends through the passageway 23 and threadably engages the threaded bore 21 of the implant 20. The screw 26 thereby secures the abutment 22 to the implant 20.

The abutment 22 further defines a second threaded bore 28. The bore 28 is oriented at an angle within the abutment, so that upon connection of the abutment 22 to the implant, the bore 28 is disposed at a substantially vertical orientation (±5°). Accordingly, implementation of the present invention provides a plurality of angled abutments 22 (see FIG. 1 and abutments 22a-d) with bores 28 at differing angles, for selective installation. In an alternate embodiment, the abutment 22 defines opposing base surface and seating surface with a threaded bore through the seating surface into the abutment. The base surface is defined at various angles to offset the angle of the installed implant member, so that the seating surface is substantially horizontal for receiving a fastener into the bore at a substantially vertical orientation.

The rail 14 secures with the clip assembly 24 to the abutment 22. The clip assembly 24 includes a lower clip 30, an opposing upper clip 34 and an assembly locking member 36. The rail 14 is held between the lower clip 30 and the upper clip 34, as discussed below. In the illustrated embodiment shown in FIG. 2, each clip assembly 24 further includes a spacer column 32. The spacer column 32 defines an axial passageway 37. In the illustrated embodiment, the spacer column 32 defines a threaded distal end 38. The securing member 36 is a nut threadably received on a threaded distal end 38 of the spacer column 32. A clip assembly screw 40 extends through the nut 36, the open end of the spacer column 32, and through the passageway 37, and engages the threaded bore 28 in the abutment 22. The clip assembly screw 40 attaches the clip assembly 24 to the abutment 22. The angled bore 28 in the abutment 22 provides for gross correction to vertical from the angulation of the implant 20 for receiving the clip assembly 24 that holds the rail 14. The rail 14 is secured between the opposing lower clip 30 and the upper clip 34.

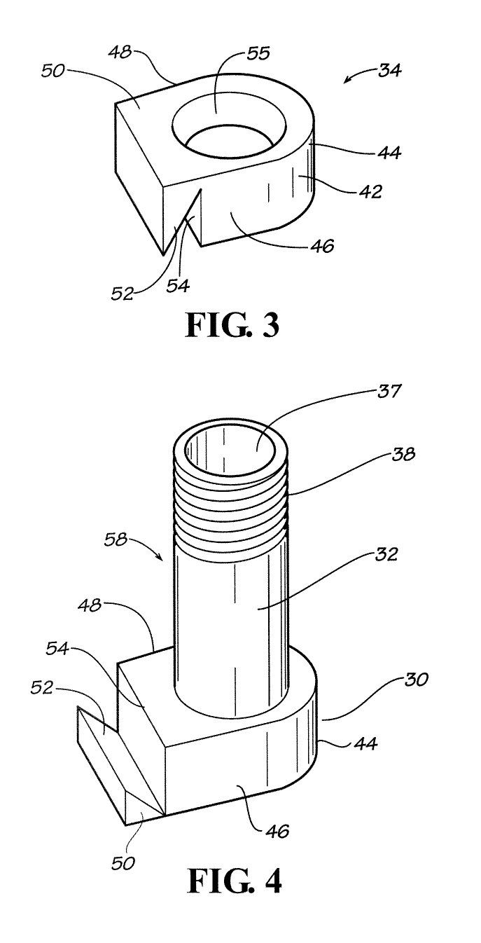

FIG. 3 is a detailed prospective view of an embodiment of the upper clip 34. The upper clip 34 comprises a member 42 having an arcuate wall portion 44 and opposing planar sides 46, 48. A lip 50 extends laterally. The lip 50 is configured for receiving an edge of the rail 14. In the illustrated embodiment, the lip 50 projects laterally from the member 42 and defines a beveled face 52 facing an abutment wall 54. The member 42 defines a through passage 55. The lower clip 30 in one embodiment has the same structure as the upper clip 34. In this embodiment of the clip assembly 34, the spacer column 32 is a separate member received in the passageway of the lower clip 30.

FIG. 4 illustrates an alternate embodiment of the lower clip 30 and the spacer column 32 as a unitary member 58. The spacer column 32 extends from the lower rail clip 30 and defines the through passageway 37. The distal end 38 of the spacer column 32 is threaded. The spacer column 32 is manufactured in selected lengths, so as to position an upper end thereof proximate, or slightly above, the surface of the gingiva tissue of a patient.

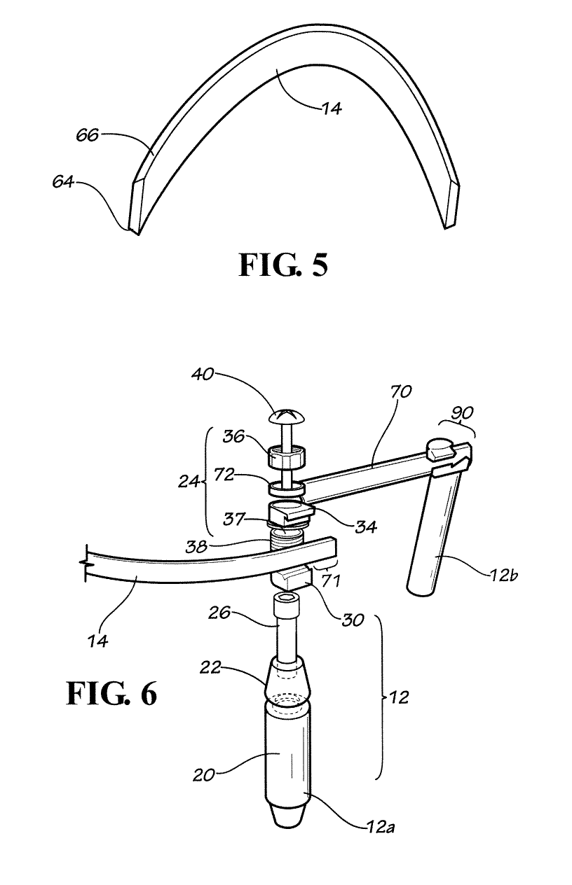

FIG. 5 is a prospective view of the rail 14 for the full arch assembly 10. The rail 14 is an elongated metal band having opposing edges 64, 66. The edges 64, 66 in the illustrated embodiment are beveled for mating contact with the respective beveled surface 52 of the opposing lower and upper clips 30, 34.

With reference to FIG. 2, the securing member 36 or nut threads on the upper end of the spacer column 32. Tightening the nut 36 secures the rail 14 rigidly between the lower clip 30 and the upper clip 34. In an alternate embodiment, a portion of the rail 14 seats on the lower clip 30 and the clip assembly screw 40 secures the upper clip 34, the rail, and the lower clip to the abutment 22.

FIG. 6 is a prospective exploded view of a dental prosthesis using the rail 14 for the anterior dental arch and a posterior rail 70 along the pre-molar and molar region of the arch. The rail 70 extends between an anterior implant assembly 12a and a posterior implant assembly 12b . The rail 14 extends arcuately anterior of the incisors and terminates with a cantilever portion 71 longitudinally beyond the anterior implant assembly 12a . In this embodiment, one end of the rail 70 includes a ring 72. The ring 72 seats on the upper rail clip 34 concentric with the passageway 37 of the clip assembly 34 for the anterior implant assembly 12a . The rail 70 extends to the posterior implant assembly 12b and may also include a posterior cantilever portion 90 thereof

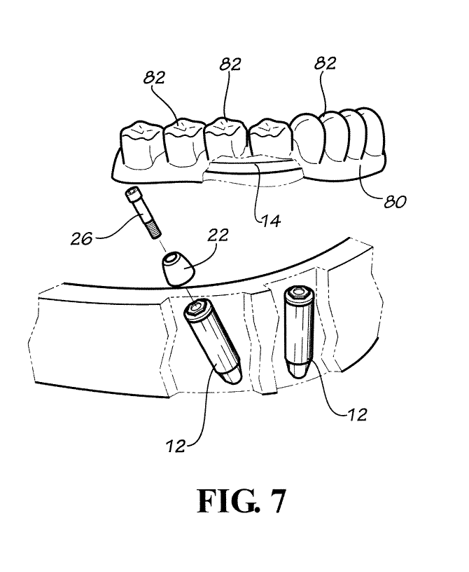

FIG. 7 illustrates a denture 80 molded of a conventional material to which a plurality of teeth 82 attach. The denture 80 defines a U-shape in cross-section for arching over a maxillary or mandibular ridge of a patient. A shown in partial cut-away view, the rail 14 seats within the arch of the denture 80.

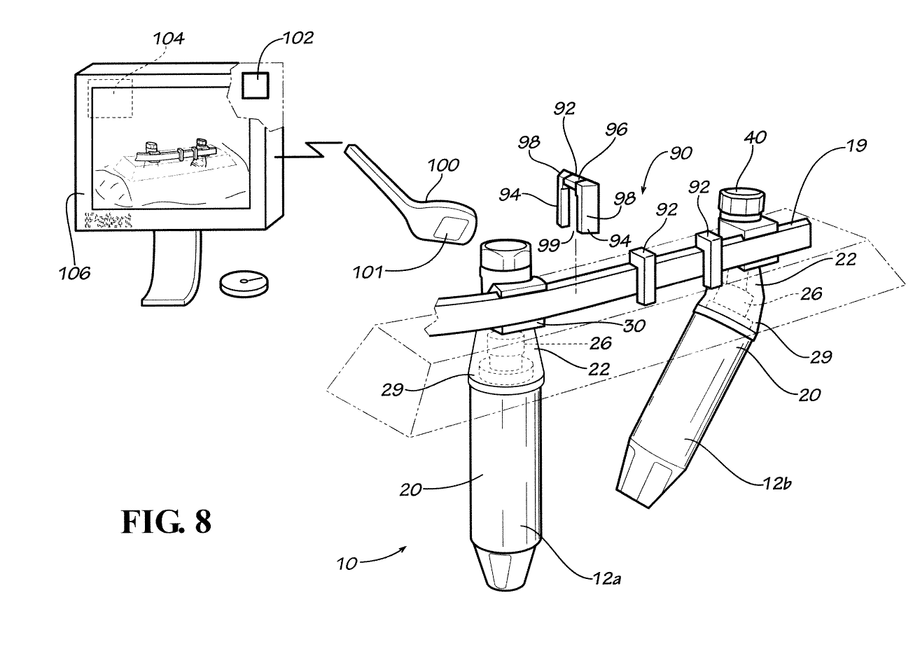

FIG. 8 illustrates a detailed partial view of the dental prosthesis 10 configured for facilitating digital imaging of a patient's mouth and soft/hard tissue for restoration surgery. The rail 14 includes a differentiation characteristic generally 90 as a marker media for use as reference points during digital imaging and manipulation of resulting images of the patient's mouth and gingiva structure for restoration surgery. In the illustrated first embodiment, the differentiation characteristic 90 comprises a plurality of members 92 that attach to the rail 14 in spaced-apart relation. The members 92 are U-shaped with opposing legs 94 and a transverse leg 96. The legs cooperatively define a gap 99 that that grippingly sliding receives a portion of the rail 14. The legs 94, 96 may further define angled surfaces generally 98. An imaging device 100 such as a CCD dental camera with a CCD device 101 operates to scan or image selectively the lingual, occusal, and buccal surfaces of the gingiva and teeth. The imaging device 100 communicates the images to an image database 102 maintained by a computer software application 104 configured to operate on a computer 106 to image, store, display and analyze the scanned images. The imaging device 100 may be connected by a cable or wirelessly for communication with the computer 106.

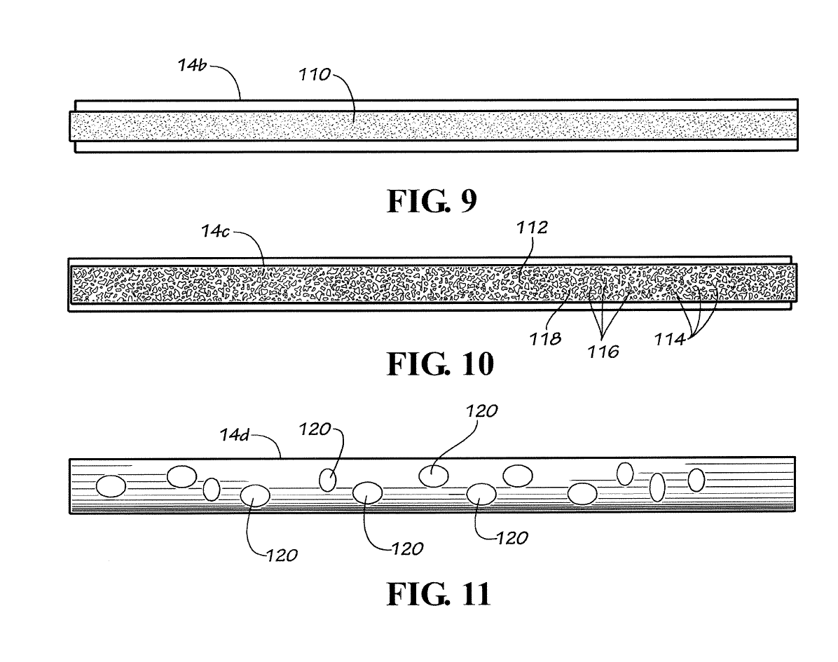

FIG. 9 illustrates a second embodiment of a rail 14b with an irregular differentiated surface 110 for defining reference points for digital imaging of a patient's mouth and gingiva structure for restoration surgery. The surface 110 may be a graphic image that is painted, etched, applied, or attached as a separate member to the rail 14b , including the lingual, upper and buccal sides of the rail. The graphic image that defines the surface 110 provides different elements therealong. The distinctive and varied elements in the graphic image facilitate the compiler in overlapping of multiple images to create the composite image. An image made of a selected portion of the patient's mouth including the rail 14b may be compared and overlapped with a subsequent adjacent image by alignment of corresponding distinctive elements in the surface 110.

FIG. 10 illustrates a third embodiment of a differentiation rail 14c with an irregular texture field 112 for defining reference points for digital imaging of a patient's mouth and soft/hard tissue structure for restoration surgery. The irregular texture field 112 is defined by a plurality of spaced-apart bumps 14 or by alternating recesses 116 and peaks 118 molded or impressed into the rail 14c.

FIG. 11 illustrates a fourth embodiment of a differentiation rail 14d that defines a plurality of shaped openings 120 along the rail. The openings 120 define reference points for digital imaging of a patient's mouth and soft/hard tissue structure in restoration surgery. The openings 120 may be symmetrical or symmetrical and spaced apart evenly or varied spacing, along the rail. Further, the openings 120 may receive therein denture materials when the denture 80 is molded with the rail 14d seated within the arch of the denture, as discussed above.

With reference to FIG. 1, the dental prosthesis 10 is assembled and installed chair side for a patient. Briefly and in summary, the implant assemblies 12 are selectively positioned in selected locations of the maxillary bone structure of the patient. The denture (pre-configured) is placed in the patient's mouth on the abutments 22 on the respective distil end of the implants 20. Each of the screws 26 extends through a respective abutment 22 and threadedly engages the dental implant 20 to fixedly secure the abutment 22 to the dental implant. The lower clip 30 and spacer column 32 (separate members or unitary) are positioned on the abutment 22. The hollowed out denture 80 is seated over the assemblies 24 and the rail 14. The upper clip 34 is positioned on the threaded end of the clip member 32 so that the beveled surfaces 52 of the lower and upper clips 30, 34 are in opposing facing relation. The nut 36 is loosely threaded on the threaded end 38 of the spacer column 32. The rail 14 seats so that the beveled edges 64, 66 contact the beveled surfaces 52 of the opposing lower clip 30 and upper clip 34. The screws 40 are tightened to secure the clip assemblies to the abutments and hold the rail 14.

In greater detail, the assembly and installation of the dental prosthesis 10 is described below. First, prior to chair side assembly of the dental prosthesis 10 in accordance with the present invention, a conventional denture is made. This involves taking an impression or casting of the patient's maxillary or mandibular ridge that will receive the dental prosthesis 10 as a bridge or replacement denture. As shown in FIG. 1, this may involve a full denture, while an alternate embodiment may use implant assemblies 12 with the rail 14 mounted there between as a bridge along a portion of the dental arch where several teeth may be missing, for example, the incisors portion of mandibular jaw. The denture is fabricated conventionally with porcelain or acrylic teeth embedded in denture material. Further, x-ray, photographic, or digital images are made of the patient's alveolar structure. The oral surgeon analyzes the images of the alveolar bone structure and determines a suitable location and angular orientation of the implant assemblies 12. For example, the upper jaw has a sinus cavity that should be avoided while the lower jaw has nerves that likewise need to be avoided.

The implant assemblies 12 are placed conventionally. This involves drilling pilot holes in the selected location and angle. The process of drilling the pilot holes provides the surgeon with information as to bone density. Angled installation of implants provides longer engagement of the implant with the bone and provides better mechanical stability. Pilot holes may be drilled with successively larger diameters. The implant 20 screws into the drilled hole in the bone structure. The length of the implant 20 is selected so that an upper end of the implant 29 is disposed proximate, at or just below, the soft tissue gingival line of the patient. The abutments 22 are then attached. The abutments 22 correct gross angulation error of the implants 20 in order to provide the substantially vertical orientation of the threaded bore 28 of the abutment. The abutment 22 is selected so as to have an end disposed projecting above the surface of the soft tissue.

The denture is prepared for fitting. An impression material is placed within the open bottom of the denture. The denture is seated on the jaw. The patient then bites down firmly. Each of the abutments 22 makes an indention in the impression material. The denture is removed. The point of contacts of the abutments 22 are drilled through the denture. This creates an opening for access to the abutment. An opening in the denture is made for each implant.

The rail 14 is then adapted to the denture 80. It may be necessary to grind the denture from below to define a channel for the rail 14 within the interior arch of the denture as shown in FIG. 8. With continuing reference to FIGS. 1 and 2, the lower clip 30 with the spacer column 32 is positioned on the abutment 22. The denture is positioned on the alveolar ridge of the patient. This seats the lower edge 64 of the rail 14 on the lower rail clip 30. The beveled edge 64 matingly contacts the beveled face 52 and a side of the rail abuts the wall 54. (It is to be appreciated that in a first embodiment, the lower rail clip 30 and the spacer column 32 are separate members, or as illustrated in FIG. 4 may be an integrated member.)

With access through the opening in the denture, the upper clip 34 seats on the distal end of the spacer column 32. The upper edge 66 similarly matingly engages the beveled face 52 of the upper rail clip 34 and the side of the rail 14 abuts the wall 54. The nut 36 threads on the threaded end 38 of the spacer column 32. The rail 14 is thereby held between the lower clip 30 and the upper clip 34. The assembled clips 30, 34 are loosely held clamping the rail 14 between them. The clip assembly screw 40 extends through the opening formed in the denture and through the nut 36 received on the threaded end of the spacer column 32. The screw 40 extends through the passageway 37 and threadably engages the threaded bore 28 in the abutment 22, to thereby secure the clip assembly 24 to the abutment 22. This step is repeated for each of the at least one other dental implant 20 as would be the case for a bridge having two implants or additional three or more implants for a full arch reconstruction.

A liquid acrylic is poured around the opening in the denture including around the lower clip 30, the upper clip 34 and assembly locking member 36. The liquid acrylic is cured. This holds the rail 14 with the clips 30, 34. The fastener 40 is unthreaded. The assembly of the denture with the rail 14 and the clip assemblies 24 may then be removed as unitary piece. The edges of the denture may then be smoothed to abut the soft tissue of the gingiva over the alveolar ridge. The denture or bridge assembly is replaced in the patient's mouth, and the assembly is secured in place with the clip assembly screws 40. The structure of the rail 14 secured by the clip assemblies 24 to the abutments 22 provides a passive fit for the denture. The rail 14 secured by the clip assemblies does not apply force to the implants 20. The openings in the denture are closed with a curable denture material.

With reference to FIGS. 8-11, imaging of a patient's mouth and gingiva is facilitated especially during full arch, or partial arch restoration, in which a plurality of sequential teeth are missing leaving only gingiva. The gingiva and soft tissue generally lacks meaningful differentiation by which a plurality of images may be overlapped to define a detailed image and 3-dimensional model of the gingiva for restoration surgery.

Imaging may be made once the rail 14 is positioned within the patient's mouth relative to the gingiva and the rail clip assemblies are locked to the rail.

With reference to FIG. 8, the members 92 attach to the rail 14 by slidingly receiving a portion of the rail 14 in the gap 99. The members 92 are disposed in spaced-apart relation. The spacing is suitable for imaging with the imaging device 100 and subsequently overlapping of multiple images by matching reference points such that a composite image may be generated. The members 92 and the angled surfaces 98 cooperatively define the reference points on which the images may be aligned for overlapping and creating the composite image. As conventional with the operation of the digital device 92 for imaging teeth (which have sufficient reference points due to variations in the surfaces of the teeth), the images may be selectively taken with a switch or may be obtained continuously in a video mode. An alternate embodiment uses a laser to emit light that reflects from the surface and is received by a receiver.

The other embodiments illustrated in FIGS. 9-11 provide alternate structures for defining reference points to be included in the images by the imaging device 100. The reference points enable the image matching system to match and overlap multiple images for creating a composite image or a 3-dimensional digital model for use in restoration surgery and creation of a denture 80. With reference to FIG. 9, the distinctive elements in the graphic image that defines the surface 110 provides reference points on which the compiler may align for overlapping of multiple images to create the composite image. With reference to FIG. 10, the irregular texture field 112 with the bumps 114 and recesses 116 and peaks 118 defining reference points for digital imaging and compilation of multiple images of the patient's mouth and gingiva structure. With reference to FIG. 11, the shaped openings 120 define reference points for digital imaging of the patient's mouth and gingiva structure and compilation of multiple images. The openings 120 may receive therein denture materials when the denture 80 is molded with the rail 14d seated within the arch of the denture.

After imaging, the rail 14 with the attached rail clips, is removed. This is accomplished by removing the fastener 40. The rail may then be received into the denture 80 as discussed above.

It is to be appreciated that the rail 14 is disposed in a horizontal plane for compliant design of a restoration whereby restoration teeth align with existing teeth and/or other restoration teeth in the upper and lower mandibular jaw. The implant assembly necessarily orients the clip assembly so that the rail 14 extending between the spaced-apart clip assemblies is horizontal. In one embodiment, the implant assembly is a unitary member configured for threadably engaging the bone of the jaw and the upper end defining an angled surface for seating the clip assembly so that the rail will be horizontal and the first and second clips hold the rail horizontal. It may be however, that the abutments are selectively attached to the implant in order to orient the clip assemblies and the rail clips properly.

The apparatus and method disclosed herein can be made and executed without undue experimentation in light of the present disclosure. While the apparatus and methods of this invention have been described in terms of illustrative embodiments, it will be apparent to those of skill in the art that variations may be applied to the apparatus and in the method steps or in the sequence of steps thereof described herein without departing from the concept, spirit and scope of the invention. All such similar substitutes and modifications apparent to those skilled in the art are deemed to be within the spirit, scope and concept of the invention as defined by the appended claims.

What is claimed is:

1. A dental prosthesis apparatus with reference points, comprising:
at least a pair of implant assemblies, each implant for selective imbedding in a maxillary bone structure and defining a threaded bore;
at least a pair of clip assemblies, each clip assembly comprising:
a first rail clip;
an opposing second rail clip; and
a fastener for securing the first rail clip and the second rail clip in spaced relation; and
a threaded member for securing the clip assembly to the implant assembly by engaging the threaded bore;
a rail extending between the pair of clip assemblies and disposed with a respective portion between the first rail clip and the second rail clip of each of the clip assemblies,
whereby the clip assembly, being secured with the threaded member connected to the implant assembly, rigidly holds the rail between the first rail clip and the second rail clip; and
the rail defining a differentiation characteristic for use as reference points for matching a plurality of images in overlapping relation for a composite image by an image compiler.

2. The dental prosthesis as recited in claim 1, wherein the differentiation characteristic comprises a plurality of indicator members selectively attached to the rail in spaced relation.

3. The dental prosthesis as recited in claim 2, wherein the indicator member comprises a U-shaped member having opposing legs and a transverse member extending between respective ends of the legs, thereby defining a gap, the member receiving a portion of the rail within the gap.

4. The dental prosthesis as recited in claim 3, wherein the indicator member defines an angled surface on at least one of the legs.

5. The dental prosthesis as recited in claim 3, wherein each leg of the indicator member defines a respective angled surface.

6. The dental prosthesis as recited in claim 1, wherein the differentiation characteristic comprises an irregular differentiated surface.

7. The dental prosthesis as recited in claim 6, wherein the irregular differentiated surface is printed on the rail.

8. The dental prosthesis as recited in claim 6, wherein the irregular differentiated surface is embossed on the rail.

9. The dental prosthesis as recited in claim 6, wherein the irregular differentiated surface is attached as a separate member to the rail.

10. The dental prosthesis as recited in claim 1, wherein the differentiation characteristic comprises an irregular texture field.

11. The dental prosthesis as recited in claim 10, wherein the irregular texture field comprises ridges and valleys in the rail.

12. The dental prosthesis as recited in claim 1, wherein the differentiation characteristic comprises plurality of symmetric or asymmetric shaped openings.

13. The dental prosthesis as recited in claim 1, further comprising:
a pair of abutments, each seated on a respective one of the implants and wherein the abutment defines an axial passageway that aligns with a threaded passageway in the implant and the threaded bore extends through the abutment; and
a pair of threaded fasteners, each one extending through the passageway of a respective abutment and engaging the threaded axial bore of the implant for securing the abutment to the implant.

14. The dental prosthesis as recited in claim 1, wherein each clip assembly further comprises a spacing member having a threaded first end and defining a longitudinally extending passageway therethrough, the threaded first end for engaging the fastener to secure the first clip and the second clip together.

15. A method of positioning reference points in a dental prosthesis apparatus for imaging of a dental patient's mouth and gingiva for restorative surgery, comprising the steps of:
(a) imbedding in a maxillary bone structure at least a pair of implant assemblies, each implant defining a threaded bore;
(b) attaching at least a pair of clip assemblies, each clip assembly attaching to a respective implant assembly, each clip assembly having a first rail clip and an opposing second rail clip;
(c) extending a rail having a differentiation characteristic between the first rail clip and the second rail clip in each clip assembly; and
(d) securing the first rail clip and the second rail clip together in the respective clip assembly to hold the rail rigidly therebetween;
whereby the rail having the differentiation characteristic, defines thereby reference points for matching a plurality of images in overlapping relation for a composite image by an image compiler.

16. The method as recited in claim 15, further comprising the step of providing the differentiation characteristic by attaching a plurality of indicator members to the rail.

17. The method as recited in claim 16, wherein the indicator members each comprises a U-shaped member having opposing legs and a transverse member extending between respective ends of the legs, thereby defining a gap, the member receiving a portion of the rail within the gap.

18. The method as recited in claim 15, further comprising the step of providing the differentiation characteristic by providing the rail with an irregular differentiated surface.

19. The method as recited in claim 15, further comprising the step of providing the differentiation characteristic by providing the rail with an irregular texture field.

20. The method as recited in claim 19, wherein the irregular texture field is defined by embossing valleys into the surface to define valleys and ridges.

21. The method as recited in claim 15, further comprising the step of providing the differentiation characteristic by providing the rail with a plurality of symmetric or asymmetric shaped openings.
